(19) United States
(12) Patent Application Publication
(10) Pub. No.: US 2025/0339298 A1
(43) Pub. Date: Nov. 6, 2025

(54) COMPLIANCE-ENHANCING BLOOD VESSEL GRAFTING

(71) Applicant: EDWARDS LIFESCIENCES CORPORATION, Irvine, CA (US)

(72) Inventor: Or Cohen, Modi'in (IL)

(21) Appl. No.: 19/271,113

(22) Filed: Jul. 16, 2025

Related U.S. Application Data
(63) Continuation of application No. PCT/US2024/013239, filed on Jan. 26, 2024.
(60) Provisional application No. 63/482,190, filed on Jan. 30, 2023.

(51) Int. Cl. A61F 2/958; A61F 2/07; A61M 25/10

(52) U.S. Cl. CPC A61F 2/958; A61F 2/07; A61M 25/10; A61F 2220/0016; A61F 2230/0008; A61M 2205/0216

(57) ABSTRACT

A method of managing blood flow involves providing a vascular implant device comprising a compliant tube, advancing the vascular implant device to a target location within a target blood vessel through a transcatheter access path, anchoring first and second end portions of the vascular implant device to the target blood vessel, at least a portion of the compliant tube being disposed between the first and second end portions, and resecting a portion of the target blood vessel around the at least a portion of the compliant tube.

### RELATED APPLICATION(S)

This application is a continuation of International Patent Application No. PCT/US24/13239, Jan. 26, 2024, which claims the benefit of U.S. Provisional Patent Application Ser. No. 63/482,190, filed on Jan. 30, 2023, the complete disclosures of which are hereby incorporated by reference in their entireties.

### BACKGROUND

The present disclosure generally relates to the field of medical implant devices, including vascular stent and graft implant devices. Delivery, anchoring, and expansion characteristics of such implant device can affect patient outcomes.

### SUMMARY

Described herein are devices, methods, and systems relating to stent/graft devices including grafts that have compliant tubular medial portions. Compliant tubular elements of graft implant devices of the present disclosure can include elastic, radially-expandable tubular balloons and/or non-circular covered stent frames. Axial end portions of graft implant devices of the present disclosure can comprise intravascular stent frames or other at least partially intravascular anchor structures. Such anchor structures can further comprise extravascular anchoring feature(s), such as anchoring arms/projections configured to puncture through the blood vessel wall and deflect axially either back towards the axial center of the implant device or away from the axial center of the implant device. The distal end of the anchor arm/projection can have a barb/spike form or tip configured to embed in the exterior/surface of the blood vessel.

Graft implant devices of the present disclosure can be implanted using a combination of intravascular (e.g., transcatheter) delivery of the implant device and minimally-invasive/keyhole surgical resection of at least a portion of the blood vessel in which the implant device is deployed. Such resection of the target blood vessel may advantageously be in the area around the compliant tubular component of the graft implant, such that removal of the blood vessel wall around such area exposes at least a portion of the compliant tube to allow for expansion and/or reshaping thereof without obstruction/interference of the blood vessel wall. That is, the resection of the blood vessel may produce a window in the chest cavity through which the compliant tube is exposed.

In some implementations, graft implants of the present disclosure comprise axially-translatable covers/tubes configurable to cover at least a portion of the compliant tube to restrict expansion/reshaping in the area of axial overlap between the compliant tube and the cover/interference tube. The interference tube can be coupled to one or both axial ends/anchors of the implant in a manner as to allow for selective axial positioning of the interference tube(s). As an example, an interference tube may be implemented with screw threads configured to mate with corresponding screw threads of a fixed structure of a given axial end portion/anchor, such as an outer tube in which the interference tube can be at least partially disposed. Processes of the present disclosure can involve selectively translating the interference tube(s) to produce an exposed portion/length of the compliant tube that produces the desired compliance functionality (e.g., volume change between high-and low-pressure conditions).

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Any of the example methods and structures disclosed herein for treating a patient also encompass analogous methods and structures performed on or placed on a simulated patient, which is useful, for example, for training; for demonstration; for procedure and/or device development; and the like The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 shows example cardiac and vascular anatomy.

FIGS. 2A and 2B show side and axial cross-sectional views, respectively, of a compliant blood vessel experiencing compliant expansion and contraction over a cardiac cycle.

FIG. 3 shows an aortic blood vessel segment with example intravascular compliance-enhancing implant locations in accordance with some examples.

FIG. 4 shows an aortic blood vessel segment with example blood vessel resection locations for compliant graft implant devices in accordance with some examples.

FIG. 5 shows a compliance-enhancing graft implant device anchored to a blood vessel segment in accordance with some examples.

FIGS. 6A, 6B, 6C, and 6D illustrate a flow diagram for a process for implanting a compliant graft implant device in accordance with some examples.

FIGS. 7, 8, 9, 10, 11, 12, 13, 14, and 15 provide images of aspects of examples of the graft implant device, delivery system components, and anatomy relating to operations of the process of FIGS. 6A, 6B, 6C, and 6D.

FIGS. 16, 17, 18, and 19 show an adjustable compliance-enhancing implant device in various configurations accordance with some examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Anatomy and Compliance

Certain examples are disclosed herein in the context of vascular implant devices, and in particular, graft implant devices comprising compliant tubular segments, wherein such implant devices are implanted/implantable in the aorta. However, although certain principles disclosed herein may be particularly applicable to the anatomy of the aorta, it should be understood that graft implant devices in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves.

FIG. 1 illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3. A wall of muscle 17, referred to as the septum, separates the left 2 and right 5 atria and the left 3 and right 4 ventricles.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior 19 and superior 18 venae cavae, carry blood back to the heart.

The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart. The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively more frequently affected by aneurysms and dissections, often requiring open heart surgery to be repaired. The transition from ascending aorta 12 to aortic arch 13 is at the pericardial reflection on the aorta. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery and the right aortic sinus likewise gives rise to the right coronary artery. Together, these two arteries supply the heart.

As mentioned above, the aorta 16 is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

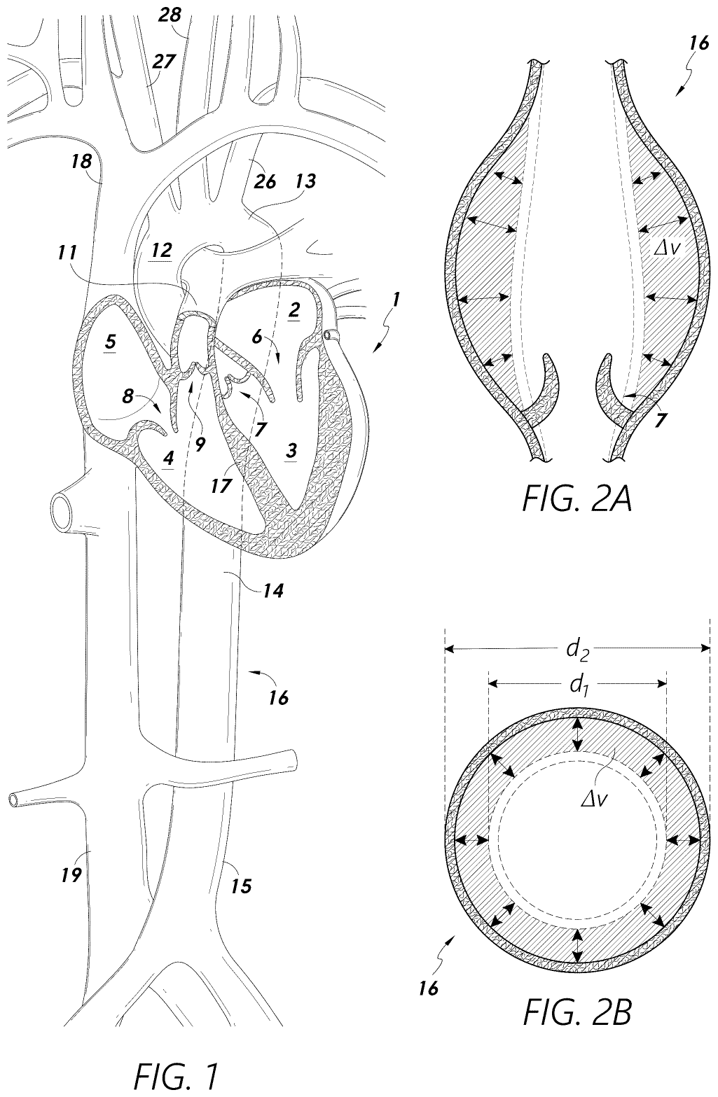

As referenced above, the systolic phase of the cardiac cycle is associated with the pumping phase of the left ventricle, while the diastolic phase of the cardiac cycle is associated with the resting or filling phase of the left ventricle. As shown in FIGS. 2A and 2B, with proper arterial compliance, an increase in volume Δv will generally occur in an artery when the pressure in the artery is increased from diastole to systole. As blood is pumped into the aorta 16 through the aortic valve 7, the pressure in the aorta increases and the diameter of at least a portion thereof expands. A first portion of the blood entering the aorta 16 during systole may pass through the artery during the systolic phase, while a second portion (e.g., approximately half of the total blood volume) may be stored in the expanded volume Δv caused by compliant stretching of the blood vessel 16 from a non-expanded diameter d₁ to an expanded diameter d₂, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where Δv is the change in volume (e.g., in mL) of the blood vessel, and Δp is the pulse pressure from systole to diastole (e.g., in mmHg):

$$C = \frac{Δ v}{Δ p} \qquad ( 1 )$$

In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

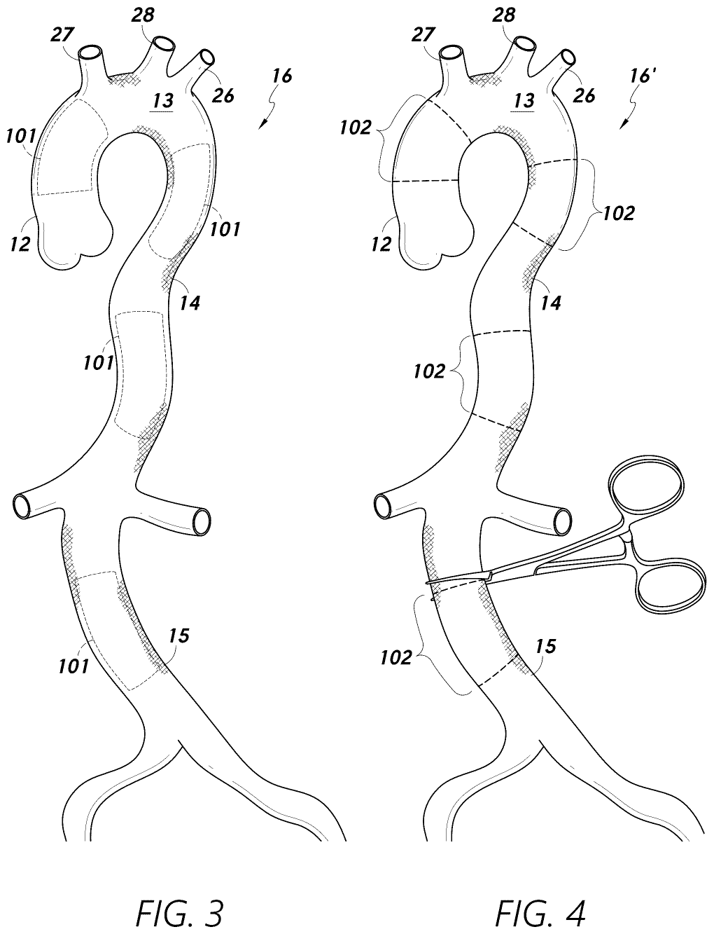

FIG. 3 shows an example stiff aorta 16′. As shown in FIG. 3, the aorta tends to change in shape as a function of age, resulting in a higher degree of curvature and/or tortuosity over time. As the vasculature of a subject becomes less elastic, arterial blood pressure (e.g., left-ventricular afterload) becomes more pulsatile, which can have a deleterious effect. For example, undesirably pulsatile arterial blood flow, such as the thickening of the left ventricle muscle and/or diastolic heart failure. Stiffness in the aorta and/or other blood vessel(s) can occur due to an increase in collagen content and/or a corresponding decrease in elastin.

With the walls of the blood vessel 16′ being resistant to stretching due to the stiffness thereof, the expansion of the blood vessel diameter from the non-expanded diameter to the expanded diameter may be limited/reduced compared to the expansion of diameter of a healthy blood vessel. A stiff aorta 16′, as blood pressure increases, may experience a small amount of expansion and volume change, or the blood vessel may be sufficiently stiff that substantially no vessel expansion takes place during systole.

Generally, the majority of aortic compliance is provided in the ascending aorta 12 with respect to healthy anatomy. Furthermore, calcification frequently occurs in the area of the ascending aorta 12, near the aortic arch 13 and the great vessels emanating therefrom. Such anatomical areas can experience relatively higher stresses due to the geometry, elasticity, and flow dynamics associated therewith. Therefore, implantation/deployment of compliance-enhancing stent devices secured to blood vessel walls using circularizing support devices of the present disclosure can advantageously be in the ascending aorta 12 in some cases. While relatively less calcification tends to occur in the descending 14 and abdominal 15 aorta, implant devices of the present disclosure can advantageously be implanted/deployed in such areas as well for the purpose of increasing compliance in the aortic system.

Examples of the present disclosure provide graft implant devices that may be implanted intravascularly, at least initially, wherein such implants have compliant tube components. Such intravascular deployment can be performed in one or more locations in a compromised aorta and/or other vessel(s). For example, FIG. 3 shows example positions 101 of intravascularly-deployed graft implant devices in various potential areas of the aorta 16′. Implantation of certain graft examples disclosed herein involve blood vessel resection after intravascular implantation to reduce interference with expanding/reshaping compliant tubular components of the graft implant. FIG. 4 shows an aortic blood vessel segment 16′ with example blood vessel resection areas 102 in accordance with some examples.

### Compliance-Enhancing Graft Implants

As described above, as the vasculature of a subject becomes less elastic, arterial blood pressure (e.g., left-ventricular afterload) can become more pulsatile, which can have deleterious effects, including thickening of the left ventricle muscle and/or diastolic heart failure. Disclose herein are surgical and minimally-invasive techniques for replacing part of a stiffened blood vessel, such as the aorta, with a graft device comprising a compliant (e.g., elastically expandable) tube. For example, the present disclosure relates to tubular graft implant devices and associated processes for delivering and implanting such implant devices in anatomy, such as vasculature, of a patient. Graft implant devices disclosed herein can include tubular balloon or stent structures configured to add-back and/or increase compliance in the aorta or other arterial (or venous) blood vessel(s) to provide improved perfusion of the heart muscle and/or other organ(s) of the body. For example, example graft implant devices of the present disclosure can include expandable and/or reshapable tubes that, when implanted, are configured to increase in cross-sectional area/volume during high-pressure conditions, such as systole, and decrease in cross-sectional area/volume during low-pressure conditions, such as diastole, which serves to force blood through the target blood vessel segment by pushing the blood through the vessel as the tube volume reduces in connection with tube contraction induced by cyclical drops in blood pressure.

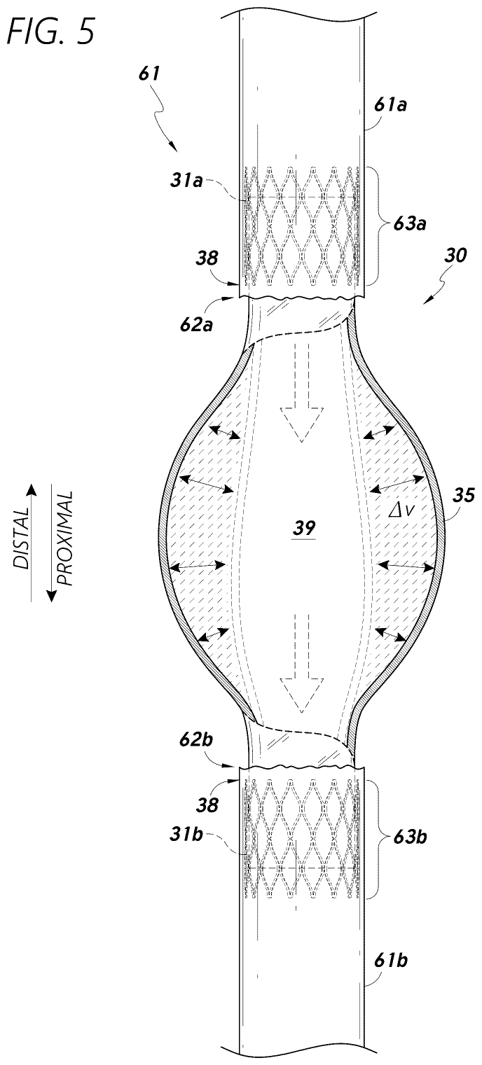

FIG. 5 shows a compliance-enhancing graft implant device 30 anchored to a blood vessel segment 61 in accordance with some examples. The blood vessel segment 61 may represent a segment of a stiffened aorta or other blood vessel. Once the device 30 is implanted in the blood vessel 61, a portion of the blood vessel wall 61 may be surgically excised to allow a compliant/expandable tube 35 of the device 30 to expand to a diameter greater than that of the blood vessel 61 to increase compliance of the blood vessel 61. The device 30 can be used to manage blood flow in a target blood vessel.

The compliant tube 35 may comprise an elastic balloon tube or other structure configured to change in cross-sectional area or volume between high-and low-pressure phases of the cardiac cycle to facilitate perfusion. As described above, relatively non-compliant blood vessels generally may not be able to stretch to thereby increase the perimeter of the blood vessel in response to increased pressure conditions. Such inability to stretch can prevent compliant expansion of the blood vessel 61. Using expandable/reshapable tube components as blood vessel grafts can increase compliance in a target blood vessel.

Although described as elastically-expandable balloon tubes in some contexts herein, it should be understood that compliant tubes of the present disclosure can comprise covered frames that are configured to reshape from more-circular axial cross-sectional shapes to less-circular axial cross-sectional shapes, which can provide for compliant volume change without necessarily requiring elastic expansion. For example, for a non-stretchable tube, generally, the greatest area/volume of the tube may be present/achieved when the tube forms a circular cross-sectional shape. Diverging from a circular cross-sectional shape can produce a cross-sectional area/volume for a tube that is less than the maximum, circular area. Therefore, transitioning a tube from a more-circular shape to a less-circular shape can provide a reduction in area/volume of the tube, and therefore solutions that utilize compliant tubes that are configured to transition between more-circular and less-circular (e.g., oval) shapes between cardiac phases can provide compliance characteristics without the need for elasticity in the tube. Any graft example described herein as including an elastically stretchable/expandable tube component can be understood to possibly be implementable using a tube that changes shape between more-circular and less-circular shapes as an alternative to, or in addition to, stretching and expanding/increasing with respect to a perimeter thereof. For example, covered tubular frames may be implemented, wherein the frame has a non-circular (e.g., oval) biased cross-sectional shape, which may be implemented using shape-memory/superelasticity characteristics of the frame; increases in luminal pressure in the tube can overcome the non-circular shape bias to circularize the tube and thereby produce an increase in volume of the tube.

The graft implant device 30 may include certain anchoring portions 31 on distal 31a and proximal 31b ends thereof; although the anchor 31a is described as ‘distal’ and the anchor 31b is described as ‘proximal,’ it should be understood that the anchor 31a may be considered ‘proximal’ and the anchor 31b considered ‘distal’ in some contexts. The tube 35 may be coupled to and/or integrated with the anchors 31 at the respective ends of the implant device 30. In some implementations, the anchors 31 comprise stent frames that are configured to be expanded within the blood vessel 61 to secure the implant 30 to the blood vessel. The term “stent” is used herein in accordance with its broad and ordinary meaning and may refer to any device configured to be implanted in a lumen of a blood vessel, the device having a tubular form forming a lumen through which blood can flow.

The anchors 31 may advantageously be implemented in a manner as to allow/provide for fluid sealing between the anchors 31 and the blood vessel 61, such that blood within the blood vessel 61 cannot pass on an outside of the anchors 31 and tube 35. The anchors 31 may be disposed at end portions of the implant 30, as shown. For example, the end portions of the implant may be considered the end quarters of the length of the implant 30, or lesser end lengths of the implant. In some contexts, the end portions of the implant 30 may be considered the portions of the implant 30 axially outside of the inner boundaries 38 of the anchors 31. The medial portion of the implant may be considered the axial segment/portion of the implant between the anchors 31, wherein the balloon tube 35 may be exposed/free between the anchors 31 in the medial segment/portion, such that the tube 35 can radially expand in such segment when not blocked/interfered with by another structure, such as the blood vessel wall or other structure of the implant 30.

The tube 35 may be coupled on an inner diameter of the stent frame anchors 31, and/or on outer diameter thereof. In some implementations, the tube 35 may be sutured, adhered, welded, crimped, or otherwise secured, to the anchors/end-portions 31. Although the implant 30 is illustrated as having anchor frames 31 for anchoring to the blood vessel 61, in some implementations, the implant 30 may not comprise separate anchoring structures/components. For example, the tube 35 may be sutured or otherwise coupled directly to the blood vessel in some implementations Such suturing may be implemented from within the blood vessel 61 using transcatheter instrumentation or other means/mechanism.

The balloon tube 35 may be configured to radially expand, as shown in the cutaway cross-sectional side view in FIG. 5. As blood within the channel 39 of the tube 35 increases in pressure, such pressure increase may produce hoop stress in the walls of the balloon tube 35, thereby producing elastic stretching of the tube 35 to increase the volume of the tube 35 and store elastic energy in the walls of the tube 35. Such energy may be returned to the blood circulation as pressure levels in the channel 39 decrease, thereby permitting the stretched tube walls 35 to contract to their relaxed/biased smaller-diameter shape, thereby reducing the volume of the channel 39 and pushing blood through the implant 30.

The implant 30 may be implanted intravascularly, wherein the anchors 31 and tube 35 may at least initially be disposed within a segment of the target blood vessel 61. After transcatheter intravascular deployment, cuts/excisions 62a, 62b may be made in the blood vessel 61 to remove a segment thereof that otherwise would cover the balloon 35 and impede radial expansion thereof. For example, the blood vessel 61 may be a relatively stiff blood vessel segment, such that the blood vessel wall 61 may not permit substantial radial expansion. Therefore, removal/resection of the blood vessel in a segment thereof covering the balloon 35 in the medial portion of the implant may be excised/removed to permit compliant expansion of the balloon 35, thereby allowing the implant 30 to operate as a compliant graft for the blood vessel 61 and improve blood flow therein. The anchors 31 may have any suitable or desirable length. For example, the anchors/stents 31 may span respective segments 63 of the blood vessel and provide fluid sealing therewith, such that blood in the first segment 61a may enter the balloon 35 and pass into the second blood vessel segment 61b without blood leaking into the anatomical cavity outside of the blood vessel 61, even when the blood vessel in the medial portion/segment of the implant 30 has been excised/removed. Such resection/excision of the blood vessel may advantageously be performed after sealing engagement between the anchors 31 and the blood vessel 61 has been achieved through anchor expansion, suturing, and/or other means/mechanism

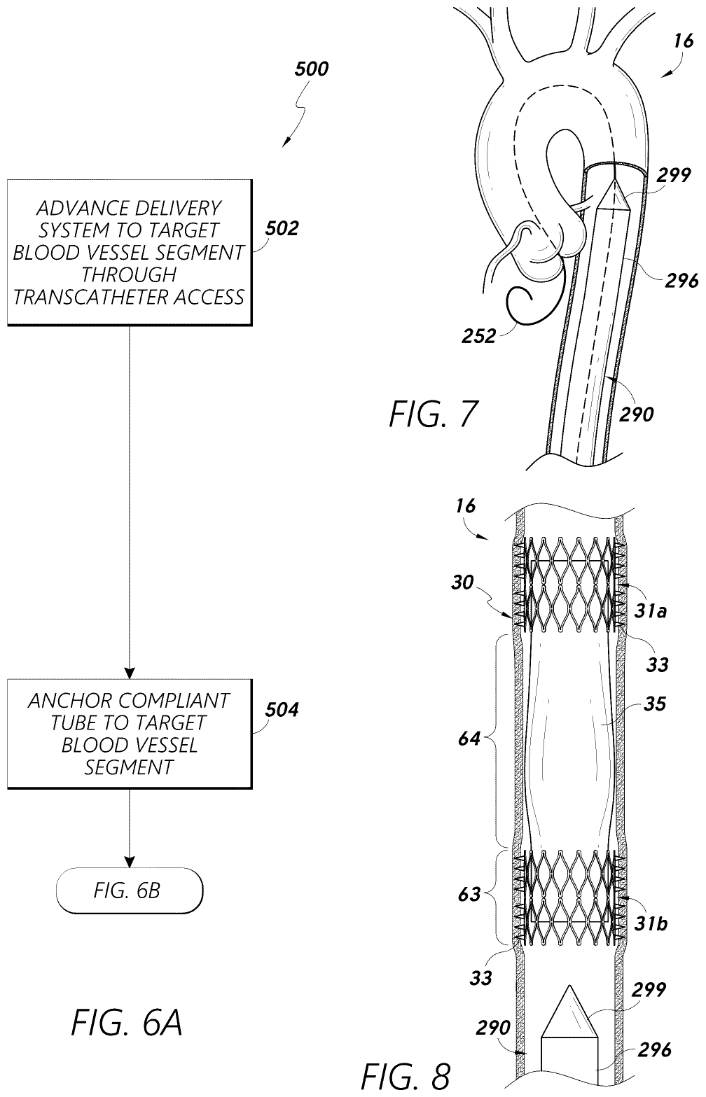

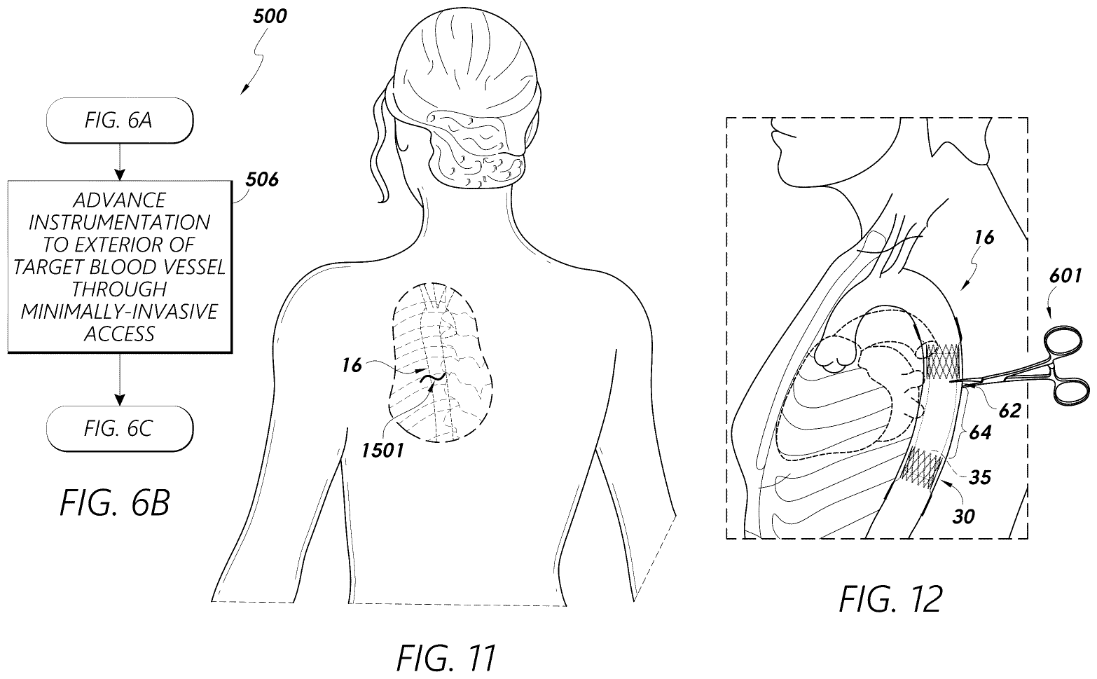

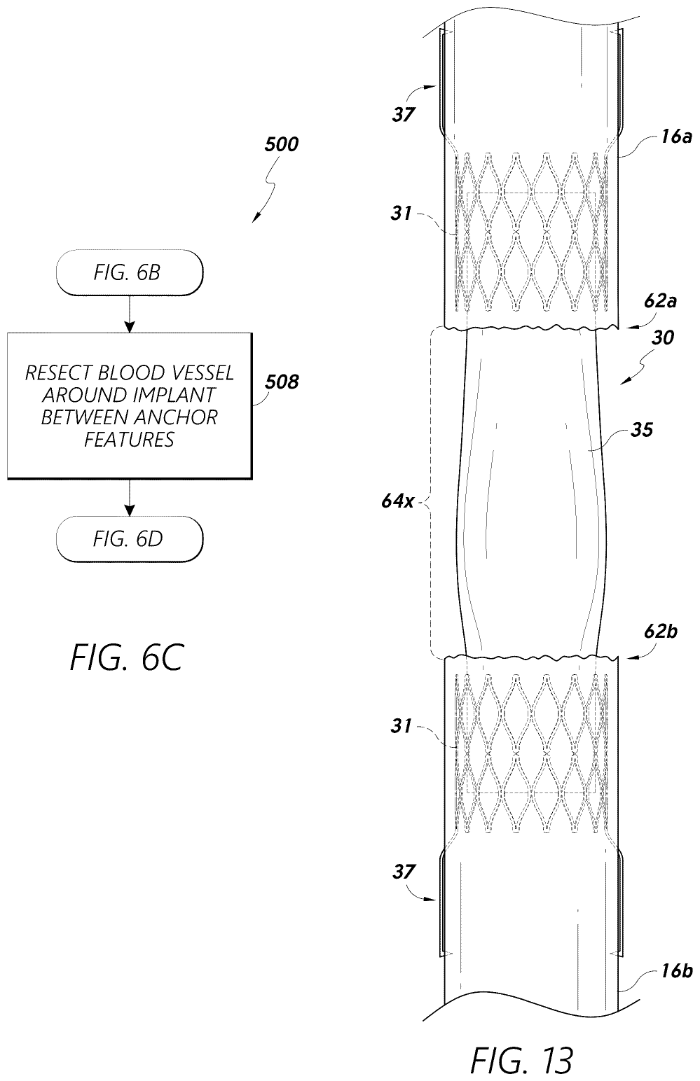

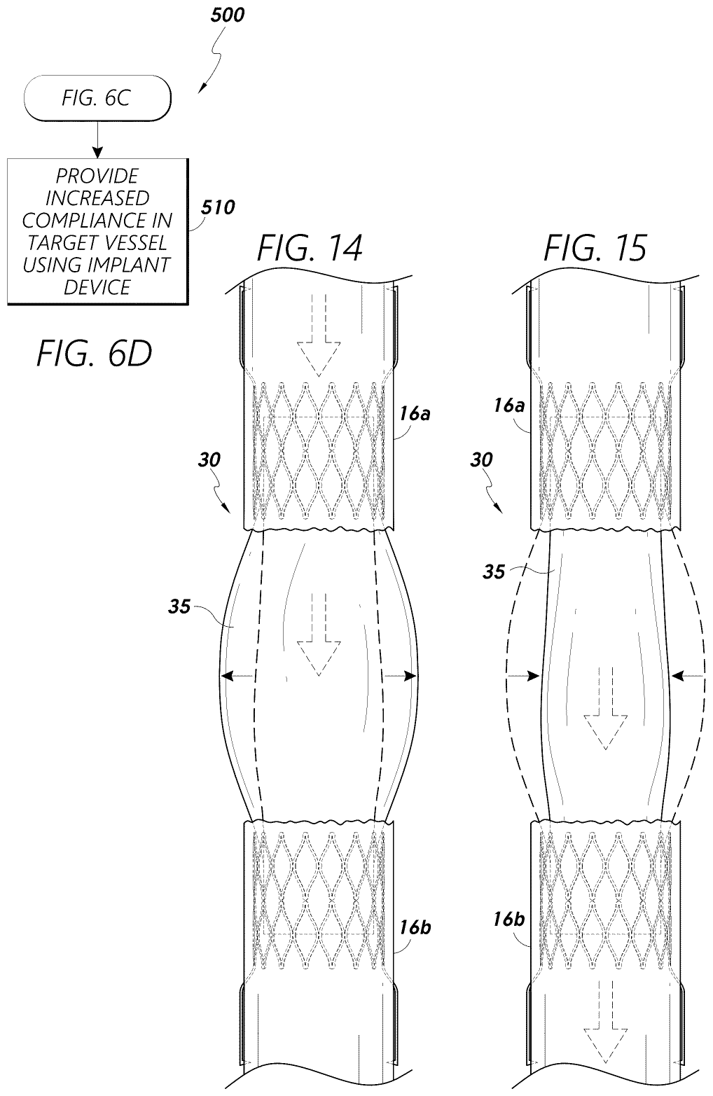

FIGS. 6A, 6B, 6C, and 6D illustrate a flow diagram for a process 500 for implanting a compliant graft implant device in accordance with some examples. FIGS. 7, 8, 9, 10, 11, 12, 13, 14, and 15 provide images of aspects of examples of the graft implant device, delivery system components, and anatomy relating to operations of the process of FIGS. 6A, 6B, 6C, and 6D Description of process 500 and blocks 502-510 can be understood with reference to FIGS. 6A-6D.

At block 502, the process 500 involves advancing a delivery system 290 to a target blood vessel segment 16 through a transcatheter access path. For example, transfemoral or other arterial access may be utilized to provide access to the target implantation site, which may be within thoracic and/or abdominal area of the aorta 16, or other aortic or blood vessel segment. FIG. 7 shows the delivery system 290, which includes one or more shafts, sheaths, catheters, and/or the like, wherein a compliance-enhancing graft implant as disclosed herein may be disposed within the delivery system 290, such as within an outer sheath/catheter 296 thereof. In some implementations, the delivery system 290 is guided to the target vascular location over a previously-deployed guidewire 252. The delivery system 290 may comprise an atraumatic leading nosecone feature 299, which may facilitate smooth passage of the delivery system 290 through the vasculature to the target location.

At block 504, the process 500 involves anchoring a compliance-enhancing implant device 30 within the target blood vessel 16. For example, the implant 30 may be anchored in a manner as to provide blood-sealing around anchor portions/elements 31 of the implant 30, thereby directing blood flow within the blood vessel 16 through inner flow channel of a compliant tube 35 of the implant 30. For at least a temporary period, some amount of blood may collect in the area outside of the tube 35 and within the blood vessel segment 64, wherein such collected blood may be stagnant for a period until removal of the blood vessel segment 64 and/or portion thereof. FIG. 8 shows the implant device 30 anchored within the blood vessel 16, as implanted using the transcatheter delivery system 290 and procedure referenced.

Anchoring the implant 30 may comprise expanding stent frame anchors 31, as shown in the example image of FIG. 8, or any other anchor structure/form. For example, such stent anchors 31 may comprise tissue engagement features 33, such as barbs, spikes, hooks, or other forms configured to embed in and/or through the blood vessel wall to thereby secure the anchors 31 to the blood vessel 16. The anchors 31 may comprise fluid-tight/impeding coverings on inner and/or outer diameters thereof, such as cloth, polymer, biological tissue, or the like, which may facilitate fluid-sealing of the anchors 31. The tube 35 may be coupled in some manner to the anchors 31, such as through suturing or other coupling means/mechanism. In some implementations, the implant 30 does not include the stent anchors 31, but rather the process 500 may involve directly coupling, such as through suturing, the compliant tube 35 to the blood vessel 16 to seal the tube 35 to the blood vessel 16.

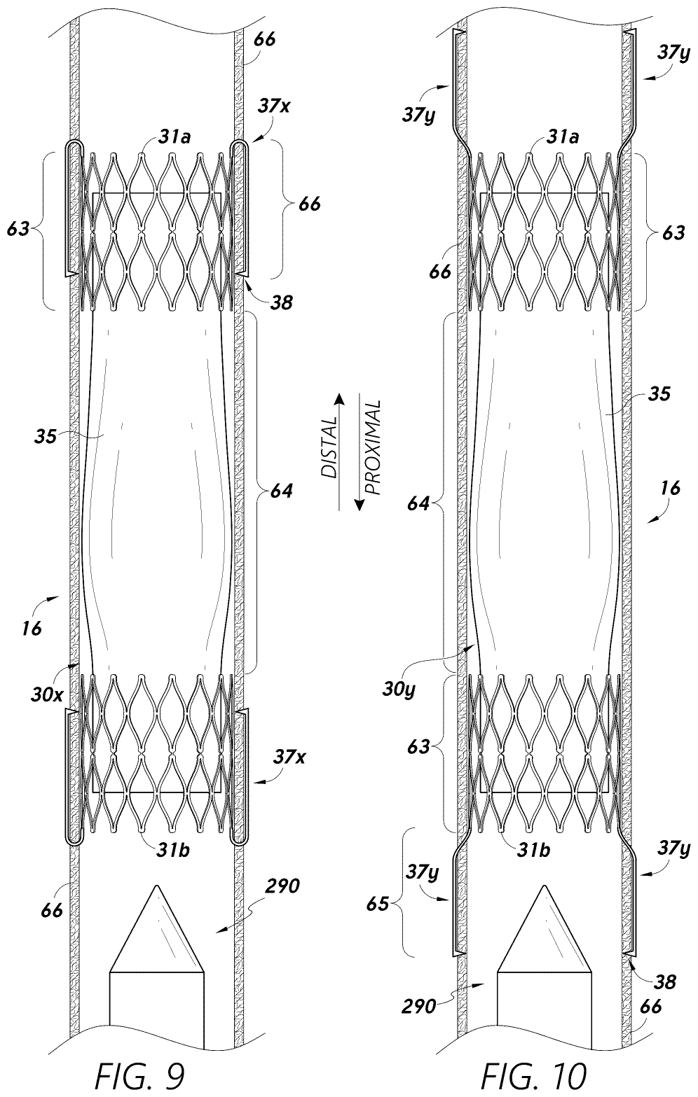

The tube 35 may be anchored using barbs that project through the target blood vessel, such as from support structures/frames 31 at the ends of the tube 35. The barbs may have elongated, arm-type forms that may fold back against the outside of the tube 35 and/or support structures/anchors 31, sandwiching the blood vessel against the outside of the tube 35 and/or support structures/anchors 31. Alternatively, the anchoring barbs/arms may fold to point/project axially away from the tube 35. FIGS. 9 and 10 show graft anchoring means/mechanisms that may be implemented in addition to and/or as an alternative to the barb/spike-type tissue-engagement features 33 shown in FIG. 8. In the examples of FIGS. 9 and 10, the anchors 31 have associated therewith certain anchoring projections/arms 37, which may be configured to deflect radially away from the anchors 31 in a manner as to protrude/puncture through the blood vessel wall to access an exterior thereof. The anchor arms/projections 37 may further be deflected axially to be secured against an exterior surface 66 of the blood vessel 16.

In the example of FIG. 9, the anchor arms/projections 37x are deflected back towards an axial center of the implant 30 in a manner as to double-back such that the arms 37x axially overlap with the respective anchor 31 from which they emanate. In such manner, the arms 37x and respective anchors 31 may sandwich the blood vessel segments 63 spanned by the anchors 31 between the anchor arms/projections 37x and the stent anchor(s) 31, as shown in FIG. 9. In some implementations, the anchor arms/projections 37 may include tissue-engagement features 38, such as one or more barbs, spikes, hooks, or the like, configured to be embedded in the exterior surface 66 of the blood vessel 16 to provide further engagement/coupling between the anchors 31 and the blood vessel 16.

In the example of FIG. 10, the anchor arms/projections 37y are axially deflected away from the axial center of the implant 30, such that the arms 37y span an axial segment 65 of the blood vessel 16 axially beyond/outside the respective ends of the anchor(s) 31. The distal ends of the anchor arms/projections 37y may be embedded into the vessel wall 66, or otherwise pinched or secured against the blood vessel wall.

The process 500 may further involve, in addition to the transcatheter graft deployment sub-process/procedure, accessing the exterior of the blood vessel for blood vessel resection/excision using a minimally-invasive and/or surgical sub-process/procedure. For example, after transluminally introducing and anchoring the tube implant 30 in the target blood vessel segment 16, the process 500 can involve, such as after a healing period (e.g., one or more days), laparoscopically excising the section of aorta coving the implanted tube 45 and leaving the tube implant 30 in-place. With reference to FIG. 6B, at block 506, the process 500 involves accessing the aorta (or other target blood vessel) 16 using a surgical and/or minimally-invasive access opening 1501, such as through the back or flank of the patient. In some implementations, the access site 1501 may be in the fourth, fifth, or sixth intercostal space between ribs of the patient, as shown in FIG. 11. A small incision may be made in the patient's back or side to provide access to the chest cavity, wherein blood vessel resection/excision instrumentation 601 (see FIG. 12) can be advanced through the incision. In some implementations, an introducer or other device may be utilized for access through the incision and/or for dilating the access opening.

FIG. 12 shows the cutting instrumentation 601 positioned to implement a cut/excision of the blood vessel 16 in an area/position within which the compliant tube 35 is disposed. At block 508, the process 500 involves resecting/excising a segment 64x of the blood vessel 16 in an area around the tube 35, advantageously in an area between the anchors 31. FIG. 13 shows the resected blood vessel with the exposed tube 35 in the resected area 64x. Such resecting/excising of the blood vessel 64x may be implemented using a surgical access through the chest or other anatomy of the patient. For example, a minimally-invasive cut made in the patient's chest/abdomen through which forceps or other cutting instrumentation may be passed to cut the blood vessel in the area 64 to exposed at least a portion of the compliant tube 35 in the anatomical chamber outside of the blood vessel 16, thereby providing space for expansion of the tube 35.

At block 510, the process 500 involves providing increased compliance in the target blood vessel 16 using the implanted device 30 with the lengthwise portion 64 of the blood vessel 16 removed. For example, as shown in FIG. 14, the compliant balloon 35 may be permitted to radially expand as luminal pressures increase therein, wherein as pressures drop/decrease, as shown in FIG. 15, the compliant balloon 35 may recoil/compress to a smaller-diameter biased shape thereof to thereby push blood flow through the implant 30 and blood vessel 16.

### Adjustable Compliance-Enhancement Window

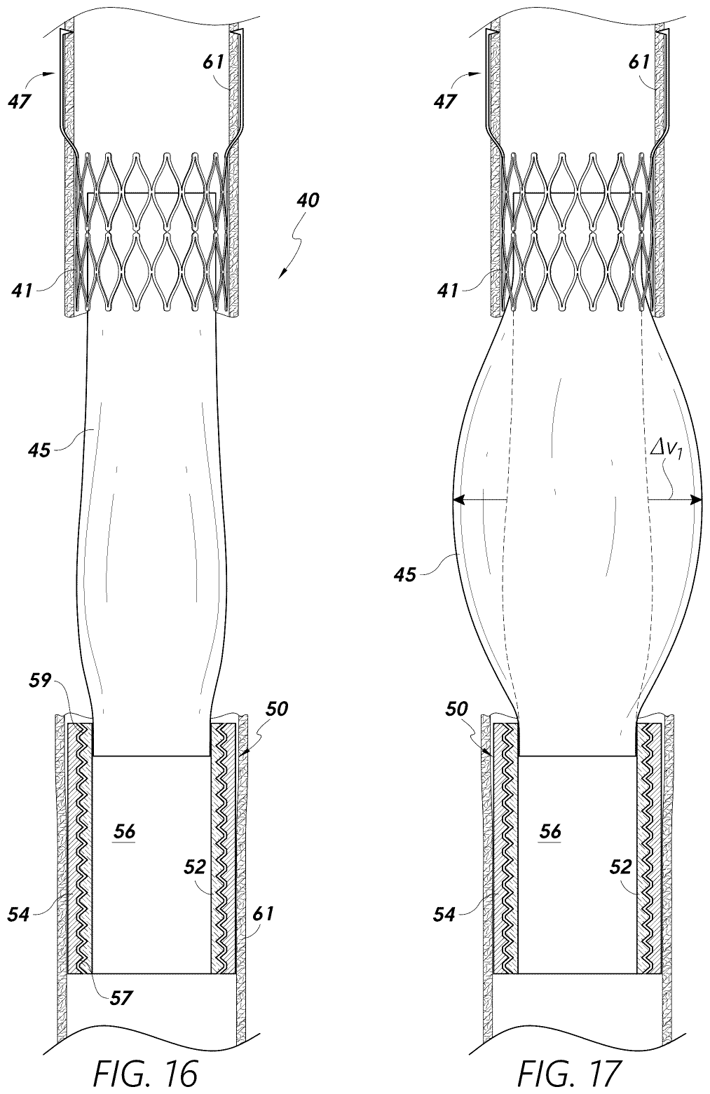

FIGS. 16, 17, 18, and 19 show an adjustable compliance-enhancing implant device 40 in accordance with some examples. The implant device 40 may be similar in any respect to any of the example compliance-enhancing graft implant devices disclosed herein. For example, the implant 40 may comprise a compliant tube 45, which may be an elastically stretchable balloon-type tube, or other volume-changing tubular device/structure. The tube 45 may be coupled at one or more ends thereof to a blood vessel anchor 41, such as a stent frame or other anchoring structure/device.

The implant 40 may further include, on one or more ends thereof, a compliance/expansion interference assembly 50, which may be integrated with and/or coupled to a blood vessel anchor/structure as disclosed herein (e.g., stent frame). For example, although the compliance-interference component/assembly 50 is shown as including an outer tube 54, and inner tube 52 without a separate blood vessel anchor coupled thereto, it should be understood that a stent frame or other blood vessel anchor may be coupled on outside or other portion of the compliance/expansion interference assembly 50 to secure the assembly 50 to the blood vessel. For example, the outer tube 54 may advantageously be secured in a fixed manner to the blood vessel 61.

The compliance-interference assembly 50 can be configured such that the inner (or outer) interference tube $2 is axially translatable relative to the fixed outer (or inner) 54 tube, such that the interference tube 52 can overlap axially at least a portion of the compliance tube 45 to restrict expansion thereof. Although the outer tube 54 is shown as a tube, in some implementations, the fixed component 54 of the interference assembly 50 may have another structure or form, such as a bar, rail, or other structure with which the interference tube 52 can be coupled in a manner such that a relative axial position between the components 52, 54 can be adjusted and/or set.

The compliance-interference assembly 50 can provide an elongatable frame that is non-or less-compliant/expandable than the tube 45. The length/segment of the assembly 50 that is coincident/overlapping with the tube 45 can be adjusted to change how much of the tube 45 is effectively compliant. The interference tube 52 of the assembly/frame 50 can be initially stored/disposed primarily within/over a rigid section/component 54 at the end of the device 40, and can be advanced by screwing or other actuation mechanism. For example, the fixed frame/component 54 can sit over an external thread 59 that is defined by one or both of the support structures 54 (e.g., on one or both ends of the implant 40) In the nested configuration of FIG. 16, the inner tube 52 is disposed primarily within the lumen/channel 58 of the outer tube 54.

The interference tube 52 may comprise certain mating features 59, such as helical threads, as shown in the illustrated example, wherein such mating features 59 are configured to mate with corresponding feature(s) 57 of the fixed component 54 of the interference assembly 50. For example, the fixed component 54 may comprise corresponding threads 57 configured to mate with the threads 59 of the interference tube 52, wherein relative rotation of the inner 52 and outer 54 components causes the interference tube 52 to axially translate relative to the outer component 54 to modify an overlap distance d; that the tube 52 projects and overlaps axially with the compliance tube 45.

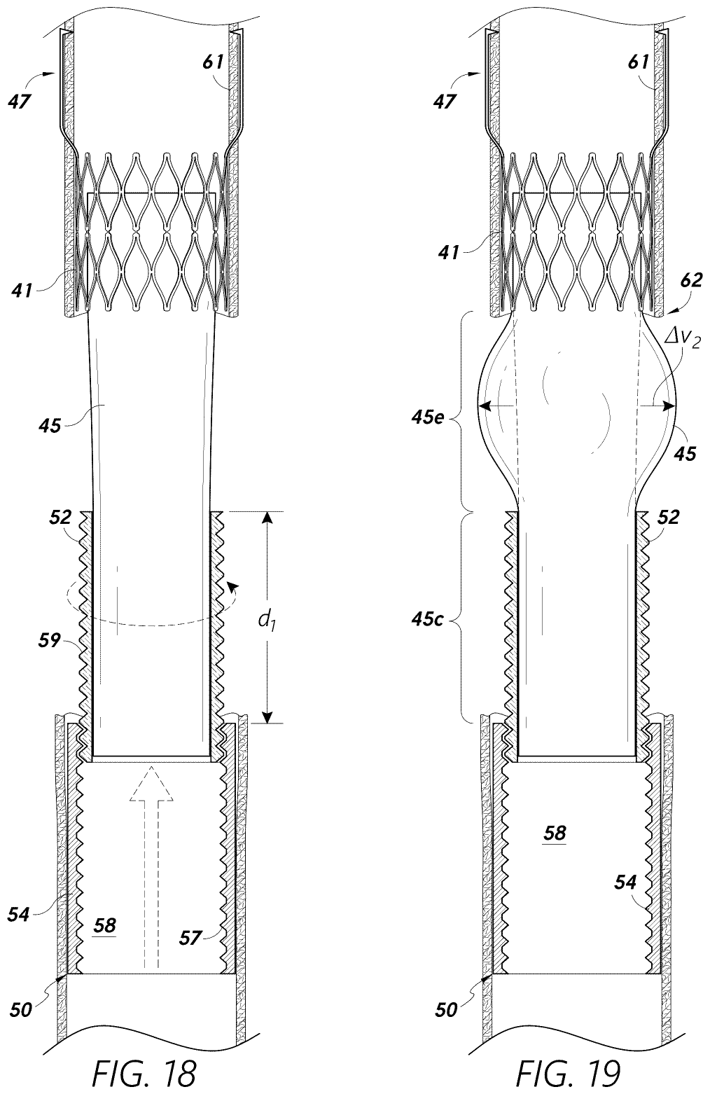

As described above, the target blood vessel 61 may be excised to provide a window/space for the balloon 45 to radially expand to thereby add compliance to the blood vessel 61. Generally, the diameter of the balloon, as well as the length of the balloon portion that is permitted to expand, can determine the amount of compliance provided by the implant 40. Therefore, by restricting the length of the balloon portion 45 that expands in high-pressure conditions, the amount of compliance provided by the implant 40 can be reduced and/or controlled to a desirable degree. For example, the interference tube 52 may be at least partially rigid, such that the diameter thereof may not increase in response to increasing luminal pressure therein, thereby restricting the ability of the tube 45 to expand in the overlapping segment d₁ (see FIG. 18).

In the configuration shown in FIGS. 16 and 17, the interference tube 52 is nested in and/or otherwise primarily overlapping with the outer fixed portion 54 of the interference assembly, such that the amount of axial overlap of the interference tube 52 with the compliant tube 45 is relatively minimal. Although FIGS. 16 and 17 show the interference tube 52 as flush with the fixed tube 54 on a axially-inner end thereof, it should be understood that such particular configuration is illustrated for example only, and the interference tube and/or inner edge thereof may be positioned at any position relative to the inner edge of the outer tube 54. With the interference tube 52 not substantially overlapping the exposed length of the compliant balloon 45, the compliant balloon 45, as shown in FIG. 17, may be permitted to expand along a substantial medial length/portion thereof in response to elevated luminal pressures without such expansion being restricted/impeded by the interference tube 52.

FIGS. 18 and 19 show a modified configuration of the interference assembly 50, wherein the interference tube 52 has been axially translated over the compliant tube 45 by a distance d₁, thereby covering the channel 56 of the tube 52 over at least a portion of the previously-exposed portion of the compliant balloon 45. As shown in FIG. 19, with the interference tube 52 projecting the distance d₁ over the compliant balloon 45, the interference tube 52 may radially cover a segment 45c of the compliant balloon 45, thereby preventing and/or impeding radial expansion of such segment 45c. Therefore, only the remaining exposed window/portion 45e of the compliance balloon 45 may be permitted to expand in a compliant manner in response to increased luminal pressure within the compliance tube 45. Therefore, compared to the expansion shown in FIG. 17, the expansion in the window 45e of the compliance balloon/tube 45 shown in FIG. 19 may be reduced, thereby decreasing the compliance effect of the implant 40. The axial segment 45e of the balloon 45 between the end/edge of the interference tube 52 and the edge 62 of the blood vessel 61 and/or anchor 41 and/or may be considered an exposure window of the compliant tube 45, as such segment may be generally/mostly open to the anatomical cavity in which the blood vessel 61 is disposed.

In some implementations, the projection distance d₁ of the interference tube 52 may be set after the implant 40 has been deployed in the blood vessel 61. For example, a transcatheter procedure may be implemented to modify the position of the interference tube 52, wherein the performance of the implant 40 may be monitored in real-time, such that the position of the interference tube 52 may be selected/modified to produce the desired result. In some implementations, the adjustment and/or setting of the position of the interference tube 52 may be performed using a surgical and/or minimally-invasive procedure as described herein.

Although the illustrated examples of FIGS. 16-19 show an interference assembly 50 associated with only one axial end portion of the implant 40, it should be understood that examples of the present disclosure may include interference assemblies associated with both axial ends of a graft implant device. For example, each such interference assembly may comprise an interference tube configured to be axially translated towards an axial center of the compliance balloon 45 and/or implant device 40.

### Additional Description of Examples

Provided below is a list of examples, each of which may include aspects of any of the other examples disclosed herein. Furthermore, aspects of any example described above may be implemented in any of the numbered examples provided below.

Example 1: A method of managing blood flow, the method comprising providing a vascular implant device comprising a compliant tube, advancing the vascular implant device to a target location within a target blood vessel through a transcatheter access path, anchoring first and second end portions of the vascular implant device to the target blood vessel, at least a portion of the compliant tube being disposed between the first and second end portions, and resecting a portion of the target blood vessel around the at least a portion of the compliant tube.

Example 2: The method of any example disclosed herein, in particular example 1, wherein said anchoring the first and second end portions is performed using a transcatheter procedure, and said resecting the portion of the target blood vessel is performed using a minimally-invasive procedure.

Example 3: The method of any example disclosed herein, in particular example 2, wherein the minimally-invasive procedure is a laparoscopic procedure.

Example 4: The method of any example disclosed herein, in particular example 2, wherein the minimally-invasive procedure is performed at least one day after the transcatheter procedure to provide a healing period.

Example 5: The method of any example disclosed herein, in particular example 1, wherein the compliant tube comprises an elastic balloon tube.

Example 6: The method of any example disclosed herein, in particular example 1, wherein the compliant tube comprises a non-circular stent frame.

Example 7: The method of any example disclosed herein, in particular example 6, wherein the non-circular stent frame has an oval cross-sectional shape.

Example 8: The method of any example disclosed herein, in particular example 1, wherein the vascular implant device further comprises a first stent frame associated with the first end portion and a second stent frame associated with the second end portion.

Example 9: The method of any example disclosed herein, in particular example 8, wherein said anchoring the first and second end portions comprises expanding the first stent frame and the second stent frame.

Example 10: The method of any example disclosed herein, in particular example 8, wherein said anchoring the first end portion comprises puncturing an anchor arm associated with the first stent frame through a wall of the target blood vessel.

Example 11: The method of any example disclosed herein, in particular example 10, wherein said anchoring the first end portion further comprises folding the anchor arm back towards the at least a portion of the compliant tube and against an outer surface of the wall of the target blood vessel.

Example 12: The method of any example disclosed herein, in particular example 10, wherein said anchoring the first end portion further comprises folding the anchor arm axially away from the at least a portion of the compliant tube and against an outer surface of the wall of the target blood vessel.

Example 13: The method of any example disclosed herein, in particular example 10, wherein a distal end of the anchor arm has a barb feature associated therewith.

Example 14: The method of any example disclosed herein, in particular example 1, wherein the first end portion of the vascular implant device is coupled to an axially translatable rigid tube.

Example 15: The method of any example disclosed herein, in particular example 14, further comprising, after said anchoring the first end portion, axially translating the rigid tube to modify an exposure window of the at least a portion of the compliant tube.

Example 16: A stent implant device comprising a first anchor associated with a first end of the stent implant device, a second anchor associated with a second end of the stent implant device, and a compliant tube disposed at least partially between the first anchor and the second anchor.

Example 17: The stent implant device of any example disclosed herein, in particular example 16, wherein the first anchor and the second anchor are stent anchors.

Example 18: The stent implant device of any example disclosed herein, in particular example 16, wherein the compliant tube comprises an elastic balloon.

Example 19: The stent implant device of any example disclosed herein, in particular example 16, wherein the compliant tube comprises a covered stent frame having a non-circular biased axial cross-sectional shape.

Example 20: The stent implant device of any example disclosed herein, in particular example 16, wherein the first anchor comprises an anchoring arm that projects from an intravascular anchor portion of the first anchor, the anchoring arm being configured to deflect towards an axial center of the stent implant device radially outside of the intravascular anchor portion.

Example 21: The stent implant device of any example disclosed herein, in particular example 20, wherein the anchoring arm comprises one or more barb features configured to embed in a blood vessel wall such that the blood vessel wall is sandwiched between a distal end of the anchoring arm and the intravascular anchor portion.

Example 22: The stent implant device of any example disclosed herein, in particular example 16, wherein the first anchor comprises an anchoring arm that projects from an intravascular anchor portion of the first anchor, the anchoring arm being configured to deflect axially away from the intravascular anchor portion.

Example 23: The stent implant device of any example disclosed herein, in particular example 22, wherein the anchoring arm comprises one or more barb features configured to embed in an outer surface of a blood vessel wall when the intravascular anchor portion is disposed within an inner diameter of the blood vessel wall.

Example 24: The stent implant device of any example disclosed herein, in particular example 16, further comprising a rigid tube that is configured to cover at least a portion of the compliant tube to limit expansion thereof.

Example 25: The stent implant device of any example disclosed herein, in particular example 24, wherein the rigid tube is configured to be axially translated to modify a radially-exposed window of the compliant tube.

Example 26: The stent implant device of any example disclosed herein, in particular example 25, wherein the rigid tube comprises first threads configured engage with second threads of the first anchor.

Example 27: The stent implant device of any example disclosed herein, in particular example 26, wherein rotation of the rigid tube causes axial translation of the rigid tube relative to the first anchor.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings

For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of managing blood flow, the method comprising:
providing a vascular implant device comprising first and second stent anchors and an elastic balloon tube disposed at least partially between axially inner ends of the first and second stent anchors, the elastic balloon tube having elasticity adapted to allow the elastic balloon tube to expand to an expanded diameter greater than diameters of the first and second stent anchors in expanded configurations thereof;
advancing the vascular implant device to a target location within a target blood vessel through a transcatheter access path;
expanding the first and second stent anchors of the vascular implant device to their expanded configurations within the target blood vessel, thereby anchoring the first and second stent anchors to the target blood vessel; and
from a position outside of the target blood vessel, resecting a portion of the target blood vessel around at least a portion of the elastic balloon tube, thereby permitting the elastic balloon tube to the expanded diameter.

2. The method of claim 1, wherein:
anchoring the first and second stent anchors is performed using a transcatheter procedure; and
resecting the portion of the target blood vessel is performed using a minimally-invasive procedure outside of the target blood vessel.

3. The method of claim 2, wherein the minimally-invasive procedure is a laparoscopic procedure performed at least one day after the transcatheter procedure to provide a healing period.

4. The method of claim 1, further comprising:
puncturing an anchor arm associated with the first stent anchor through a wall of the target blood vessel; and
folding the anchor arm back towards the at least a portion of the elastic balloon tube and against an outer surface of the target blood vessel.

5. The method of claim 1, further comprising:
puncturing an anchor arm associated with the first stent anchor through a wall of the target blood vessel; and
folding the anchor arm away from the at least a portion of the elastic balloon tube and against an outer surface of the target blood vessel.

6. The method of claim 1, wherein resecting the portion of the target blood vessel is performed using a posterior intercostal access to the target blood vessel.

7. A method of managing blood flow, the method comprising:
providing a vascular implant device comprising a compliant tube;
advancing the vascular implant device to a target location within a target blood vessel through a transcatheter access path;
anchoring first and second anchor frames of the vascular implant device to the target blood vessel at axially-offset positions within the target blood vessel, at least a portion of the compliant tube being disposed between axially inner ends of the first and second anchor frames; and
resecting a portion of the target blood vessel around the at least a portion of the compliant tube.

8. The method of claim 7, wherein the compliant tube comprises an elastic balloon tube.

9. The method of claim 7, wherein the compliant tube comprises a non-circular stent frame.

10. The method of claim 9, wherein the non-circular stent frame has an oval cross-sectional shape.

11. The method of claim 7, wherein:
the first anchor frame of the vascular implant device is coupled to an axially translatable rigid tube; and
the method further comprises, after anchoring the first anchor frame, axially translating the rigid tube to modify a radial exposure window of the compliant tube.

12. The method of claim 11, wherein axially translating the rigid tube involves rotating at least one of the rigid tube or the first anchor frame about an axis of the rigid tube.

13. The method of claim 7, wherein anchoring the first anchor frame comprises puncturing an anchor arm associated with the first anchor frame through a wall of the target blood vessel.

14. The method of claim 13, further comprising folding the anchor arm back towards an axial center of the compliant tube and against an outer surface of the wall of the target blood vessel, thereby sandwiching a wall of the target blood vessel between the first anchor frame and the anchor arm.

15. The method of claim 14, wherein a distal end of the anchor arm has a barb feature associated therewith.

16. The method of claim 13, further comprising folding the anchor arm axially away from an axial center of the compliant tube and against an outer surface of the target blood vessel.

17. A method of managing blood flow, the method comprising:
providing a vascular implant device comprising first and second end anchors and an elastic balloon tube disposed at least partially axially between the first and second end anchors, the first end anchor comprising an outer tube and a coaxial inner tube, the elastic balloon tube having elasticity adapted to allow the elastic balloon tube to expand to an expanded diameter greater than diameters of the first and second end anchors;
advancing the vascular implant device to a target location within a target blood vessel through a transcatheter access path;
anchoring the first and second end anchors to the target blood vessel;
resecting a portion of the target blood vessel around at least a portion of the elastic balloon tube, thereby permitting the elastic balloon tube to the expanded diameter; and
axially translating the inner tube of the first end anchor relative to the outer tube to cause the inner tube to radially cover a portion of the elastic balloon tube, thereby reducing a radial exposure window of the elastic balloon tube.

18. The method of claim 17, wherein the outer tube and the inner tube of the first end anchor comprise mating threads, such that rotation of at least one of the outer tube or the inner tube about an axis of the first end anchor causes axial translation of the inner tube relative to the outer tube.

19. The method of claim 17, wherein axially translating the inner tube is performed after the portion of the target blood vessel has been resected.

20. The method of claim 17, further comprising puncturing an anchor arm associated with the second end anchor through a wall portion of the target blood vessel, such that a distal end of the anchor arm is positioned radially outside of the target blood vessel.
